(19) United States
(12) Patent Application Publication
(10) Pub. No.: US 2006/0135864 A1
(43) Pub. Date: Jun. 22, 2006

(54) Peri-orbital trauma monitor and ocular pressure / peri-orbital edema monitor for non-ophthalmic surgery

(76) Inventors: L. Erik Westerlund, Solana Beach, CA (US); Christian P. Valcke, Orinda, CA (US)

Correspondence Address: FISH & RICHARDSON, PC, P.O. BOX 1022, MINNEAPOLIS, MN, 55440-1022, US

(21) Appl. No.: 11/286,312

(22) Filed: Nov. 23, 2005

Related U.S. Application Data
(60) Provisional application No. 60/631,000, filed on Nov. 24, 2004.

(51) Int. Cl. A61B 3/16; A61B 5/103

(52) U.S. Cl. 600/398; 600/587

(57) ABSTRACT

An ocular pressure monitoring system includes a plurality of transducer assemblies that measure externally applied force(s) and/or peri-orbital edema from peri-orbital tissue areas associated with one or both of a patient's eyes. Each of the transducer assemblies has at least one external force transducer and/or one edema transducer, and at least one mounting that is shaped to secure the transducer(s) to at least one peripheral peri-orbital tissue area. A microprocessor is connected to the transducers by leads or telemetrically. A display unit is in communication with the microprocessor for displaying data representative of peri-orbital edema.

This claims priority from U.S. Provisional Application Ser. No. 60/631,000, filed on Nov. 24, 2004, the entirety of which is incorporated herein by reference.

### TECHNICAL FIELD

This disclosure relates to devices, systems, and methods for monitoring peri-orbital edema as a measure of ocular and peri-orbital tissue pressure during non-ophthalmic surgery. For example, this disclosure relates to monitoring pressure on and within the eye and swelling/edema about the eye in prone or lateral positioning during non-ophthalmic surgical procedures. It further relates to devices, systems and methods of monitoring normal and tangential forces directed against the eyelids (and adjacent soft tissues) as sources of direct mechanical trauma in the same setting.

### BACKGROUND

The eyes, eyelids, peri-orbital soft tissues, and vision of a sedated or anesthetized patient are at risk to permanent injury (including blindness) during non-ophthalmic surgery. This problem has been of significant interest in recent years, and is being recognized with an increasing frequency such that one author has referred to the present and rising reported incidence of these operative complications as a “medical malpractice crisis”. A number of original scientific articles and review articles have been published in this same regard given current understanding of the interrelated effects of multiple contributing factors.

Two distinct primary categories of complication are recognized in this area of study:

1) Direct anterior mechanical trauma to peri-orbital tissues, and

2) Visual complications (to include blindness).

Direct mechanical trauma to the peri-orbital tissues is the first category of the factors that contribute to patient injury in the setting of prone surgical positioning. The most common injuries to the peri-orbital tissues are those related to unrecognized iatrogenic direct mechanical trauma to the eyelid, peripheral peri-orbital skin, or the anterior surface of the eye. These forces may be normal or tangential in vector and can result in abrasion or laceration to the upper eyelid, the lower eyelid, the skin overlying the prominence of the orbital rim, the cornea, conjunctiva, and the sclera. Mechanical insults of this nature may also result in epidermolysis, skin blistering, or full-thickness dermal injury.

The traumatic effects of these forces are compounded further by the biomechanical tissue changes that accompany ocular and peri-orbital tissue edema (that develops consequent to prone surgical positioning). The development of edema in peri-orbital tissue produces changes in the composite biomechanical compliance, tissue volume, and relational anatomy of affected tissues. This renders these tissues even more susceptible to injury from direct mechanical forces. Thus, there is a need for noninvasive methods and systems for measuring the forces that act on peri-orbital tissue during prone surgical procedures.

The second distinct category of complication that occurs in the setting of prone position non-ophthalmic surgery is indirect ocular or peri-oribital injury as manifested by visual loss and blindness. Though less common than direct mechanical trauma, visual loss/blindness in this setting is a severe and devastating complication. The peer-reviewed medical literature has identified multiple interrelated causes. The presently understood complex mechanism of interrelated factors is in distinction to a more limited prior understanding that these visual or ocular complications were secondary only to external pressure effects to the eye/globe and retinal blood flow). Although it was previously believed that external pressure on the eye was the singular factor causative of post-operative visual loss in this setting, it has since been recognized that blindness/visual loss following non-ophthalmic surgery may frequently result without the presence of external pressure to the eyes. The presently established causes are more extensive and are characteristically interrelated in a complex manner. Causative factors identified in this regard include 1) prolonged prone positioning (which produces dependent facial, ocular, and peri-orbital venous congestion and edema), 2) Trendelenberg (head-down) positioning, 3) sources of increased extra-ocular pressure causing increased intra-ocular pressure, 4) baseline intrinsic increased intra-ocular pressure, 5) general systemic hypotension, 6) low hemoglobin oxygen saturation levels, 7) increased intra-abdominal pressure, particularly during prone positioning, 8) central retinal artery thrombosis, 9) pre-existing sub-clinical retinal disease or retinal vascular disease, and others. Many of these causes have direct effects, and most of them have an indirect contributing effect on the tissue perfusion pressure to the orbital contents (to include the optic nerve and its end organ, the retina). An additional specific example of a related clinical entity is a “compartment syndrome of the optic nerve sheath”. This condition is described most in patients undergoing prone surgery, as during posterior cervical, thoracic, and lumbar spinal fusion. The syndrome manifests clinically with post-operative visual loss and/or blindness secondary to a combination of a multi-factorial perfusion injury and a mechanical traction injury to the posterior aspect of the optic nerve. Contributing factors include hypotension, low tissue oxygenation, and chemical ocular muscle paralysis as occurs during general anesthesia. The flaccid muscle state of the ocular muscles that is precipitated by general anesthesia allows the eye to subluxate anteriorly in the orbit, with gravity (in the prone position) causing further mechanical tension along the optic nerve. In addition, intra-orbital extra-ocular tissue edema (particularly of the posterior orbital adipose tissue) compounds the tension effect by both increasing circumferential optic nerve sheath pressure and further displacing the eye anteriorly in the orbit (adding still more tension to the optic nerve). The end result of this syndrome is patient blindness/visual loss secondary to a combined mechanism optic nerve injury.

Peri-orbital and/or ocular edema also produces changes in tissue size and anatomic relationship that may place the eye and its adjacent structures at risk to injury given disruption of normal passive protective mechanisms (intrinsic to the normal anatomic relationships of these tissues).

The presence or progression of many of these factors that cause ocular compromise and visual loss during prone surgery develop consequent to the progression of peri-orbital and ocular edema (and peri-orbital edema represents a common denominator and a dynamic physical manifestation of these factors).

As noted previously, the risk of ocular injury or visual loss is greatest when the anesthetized patient is in the prone (face-down) position as is often required for operations such as posterior spine fusion; spinal surgery is the non-ophthalmic surgical subspecialty of greatest documented risk with regards to ocular injury. The risk of ocular or visual complication is additionally increased when the patient is in a Trendelenberg position with the body angled head-down relative to the overall longitudinal angle of the patient as mentioned above. A further contributing risk factor is the intrinsic logistical difficulty for an anesthesia team to check or monitor the eyes of a patient in a face-down or lateral position on the operating table. This increases risk for both unrecognized direct mechanical trauma in addition to risk of visual loss and blindness from multiple other iatrogenic factors as discussed.

While the risk of upper eyelid, lower eyelid, corneal, and peri-orbital direct mechanical trauma is the most common of these complications, the risk of patient blindness after prone positioning for non-ophthalmic surgery has been recognized in the peer-reviewed published medical literature as a severe complication that is more common than previously recognized. All of these complications are problematic from a patient care standpoint, and a suitable device, method and/or system for minimizing the multi-factorial potential for peri-orbital, ocular, or visual injury is presently not available or described.

### SUMMARY

The present inventors recognized a need to improve patient safety by decreasing the potential for 1) direct eyelid and external peri-orbital trauma, and 2) ocular and/or visual complication(s) during or subsequent to non-ophthalmic surgical procedures. Accordingly, they have further recognized a need to monitor patients for risk factors in this setting. These risk factors include unrecognized external traumatic force on the upper eyelid, lower eyelid, or peri-orbital soft tissues that may result in direct trauma to these tissues. These risks also include unrecognized peri-orbital edema and ocular edema (as indicators of ocular pressure and/or decreased ocular and peri-orbital perfusion pressure) during non-ophthalmic surgical procedures.

In one embodiment, a method of monitoring a patient's eyes during a surgical procedure is disclosed. The method includes applying a first pressure sensor to a first eye of the patient, wherein the pressure sensor is applied to a peripheral superior eyelid, an inferior eyelid, or other peri-orbital tissue such that forces directed at the patient's anterior peri-orbital tissue are detected. The method further includes transmitting a first set of data representative of the detected forces to a display, and monitoring the display during the surgical procedure.

In another embodiment, a method of assessing ocular pressure includes providing an ocular pressure monitoring system, using the ocular pressure monitoring system to monitor peri-orbital edema during prone positioning of a patient, and performing a surgical procedure while monitoring peri-orbital edema.

In another embodiment, a peri-orbital trauma monitoring system is described. The peri-orbital trauma monitoring system includes a pressure sensor, a microprocessor that is capable of processing information received from the pressure sensor, communication means between the pressure sensor and micproprocessor, a display in communication with the microprocessor, and a sensor mount that attaches the sensor to peri-orbital tissue. The sensor is capable of measuring external applied loads to peripheral anterior peri-orbital tissue.

In another embodiment, an ocular pressure monitoring system includes a plurality transducer assemblies that measure peri-orbital edema from peri-orbital tissue areas associated with one or both of a patient's eyes. Each of the transducer assemblies has at least one pressure transducer and at least one mounting that is shaped to secure the transducer to at least one peripheral peri-orbital tissue area. A microprocessor is connected to the transducers by leads. A display unit is in communication with the microprocessor for displaying data representative of peri-orbital edema.

The primary objects of this invention are thus two-fold. According to one object, single or multiple point transducers are placed proximate to the anterior peri-orbital skin in a pattern specific to the measure of external sources of mechanical trauma to the skin of the superior eyelid, inferior eyelid, and peripheral peri-orbital tissue. According to a second independent object, the transducer is placed in proximity to the eye and/or peri-orbital tissues to assess tissue edema. It is a related object to provide a measure of intra-ocular and extra-ocular pressure in this setting by this approach. It is the further related object to provide a method and system for predicting retinal injury and/or visual loss/blindness due to multiple factors by measure of peri-orbital and/or ocular edema in this setting (as outlined previously). A single method and device may be used to monitor all of the above in an integrated fashion with the combination of the methods and embodiments described above.

The details of one or more embodiments are set forth in the accompanying drawings and the description below. Other features and advantages will be apparent from the description and drawings, and from the claims.

### DESCRIPTION OF DRAWINGS

These and other features and advantages will be apparent from the following more particular description thereof, presented in conjunction with the following drawings, wherein:

FIG. 1 is a diagram of one embodiment of a peri-orbital edema/ocular pressure sensor.

FIG. 2 is a diagram of a linear peri-orbital edema/ocular pressure sensor in accordance with one embodiment.

FIG. 3 is a diagram of a radial peri-orbital edema/ocular pressure sensor in accordance with another embodiment.

FIG. 4 is an illustration of an embodiment of a peri-orbital edema/ocular pressure sensor utilizing a chemically impregnated polymer.

FIG. 5 is an illustration of an axial cross section of the entire orbit and eye depicting the peri-orbital edema/ocular pressure sensor depicted in FIG. 4 applied to the eye.

FIG. 6 is an illustration of strain gage used to detect mechanical displacement in a peri-orbital edema/ocular pressure sensor.

FIG. 7 is an illustration of one embodiment of a peri-orbital trauma sensor mounting demonstrating desired peripheral placement of transducer(s) for monitoring peri-orbital trauma.

FIG. 8 is an illustration depicting a sagittal cross section of the orbit and eye/globe with the peri-orbital trauma sensor depicted in FIG. 7 applied to the eye.

FIG. 9 is an illustration of a peri-orbital edema/ocular pressure sensor mounting demonstrating desired peripheral placement of transducer(s) for monitoring peri-orbital edema.

FIG. 10 is an illustration depicting an axial cross section of the entire orbit and eye with the peri-orbital edema/ocular pressure sensor depicted in FIG. 9 applied to the eye.

FIG. 11 is a diagram of a peri-orbital edema/ocular pressure sensor system in accordance with one embodiment.

FIG. 12A is a front view of a monitor as associated with a peri-orbital edema/ocular pressure monitoring system.

FIG. 12B is a top view of the peri-orbital edema/ocular pressure monitoring system depicted in FIG. 12A.

FIG. 12C is a side view of the peri-orbital edema/ocular pressure monitoring system depicted in FIGS. 12A and 12B.

FIG. 13 is a front view of one embodiment of an eyelid pad/sensor mounting and leads for transduction of signals representing peri-orbital edema/ocular pressure.

FIG. 14 is a front view of another embodiment of an eyelid pad/sensor mounting and leads for transduction of signals representing peri-orbital edema/ocular pressure.

FIG. 15 is an illustration of two eyelid pads/sensor mountings and leads for transduction of signals representing peri-orbital edema/ocular pressure, each with a different geometry applied to a patient.

FIG. 16 is a flowchart depicting a method of monitoring for either or both of peri-orbital trauma and/or peri-orbital edema/ocular pressure during a surgical procedure.

### DETAILED DESCRIPTION

In order to provide a clear and consistent understanding of the specification and claims, including the scope to be given such terms, the following definitions are provided.

Orbit—the entirety of the osseous cavity that contains the eye as the adjacent tissues, muscles, neurovascular and connective tissue; the eye socket in layman's terms

Peri-orbital—of or pertaining to the entirety of the area or contents within or directly anterior to the osseous orbit/eye socket

Eye—the eyeball, the globe itself

Ocular—of or pertaining to the globe/eye

Extra-ocular—Outside the confines or structure of the eye/globe

Intra-ocular—Within the structure eye/globe specifically.

It has been determined that forces directed to the areas circumferentially peripheral to the eye are of significant interest. As the entirety of the orbital contents may behave as a semi-liquid medium within the confines of the osseous orbit, the contents of the osseous orbit may act as a pressure transfer medium with application of external force(s). Thus, measure of external normally-directed forces applied to these (non-ocular) peripheral peri-orbital areas can provide an indirect measure of intrinsic extra-ocular tissue pressure within the rigid confines of the osseous orbit (to include the optic musculature, the optic nerve, the lacrimal nerve, the nasociliary nerve, peri-orbital adipose tissue, and the related neurovascular supply to these structures, and (indirectly) the eye itself). Described herein are methods and systems that provide an indirect means of assessing potential perfusion pressure compromise of these peripheral peri-orbital tissues, and also provide a previously undescribed means of indirectly assessing intra-ocular and extra-ocular pressure, optic nerve pressure, and disruption of retinal perfusion pressure (through measures intentionally performed in areas removed from the eye itself at the periphery of the osseous orbit). This is in distinction to previously described methods in this regard, which have been directed with specific interest in the anterior eye/globe or the region of the superior eyelid immediately overlying the anterior eye (for purposes such as glaucoma testing, nerve injury monitoring, etc).

The present invention therefore provides methods and systems for identifying active sources of potential compromise to the eye, superior and inferior eyelids, extra-ocular peri-orbital tissues and vision by action of either or both of two complimentary methods for use during non-ophthalmic surgery, particularly in the prone position. In one embodiment, the intended application of these methods is directed to the tissues anterior to and within the (osseous) orbit, as appropriate to the unique and complex nature, mechanism, and physiology of factors that effect vision and general eye function in the setting of prone patient positioning.

The first method described involves a system for monitoring against application of unrecognized forces to the upper eyelid, lower eyelid, lacrimal duct, and the soft tissue margins more peripheral to the eye (to include the soft-tissue area directly overlying the orbital rim). This is performed to identify unrecognized sources of direct mechanical trauma to these structures as may occur during prone surgical positioning. The traumatic force(s) may be sharp, shear, blunt, or combined in nature.

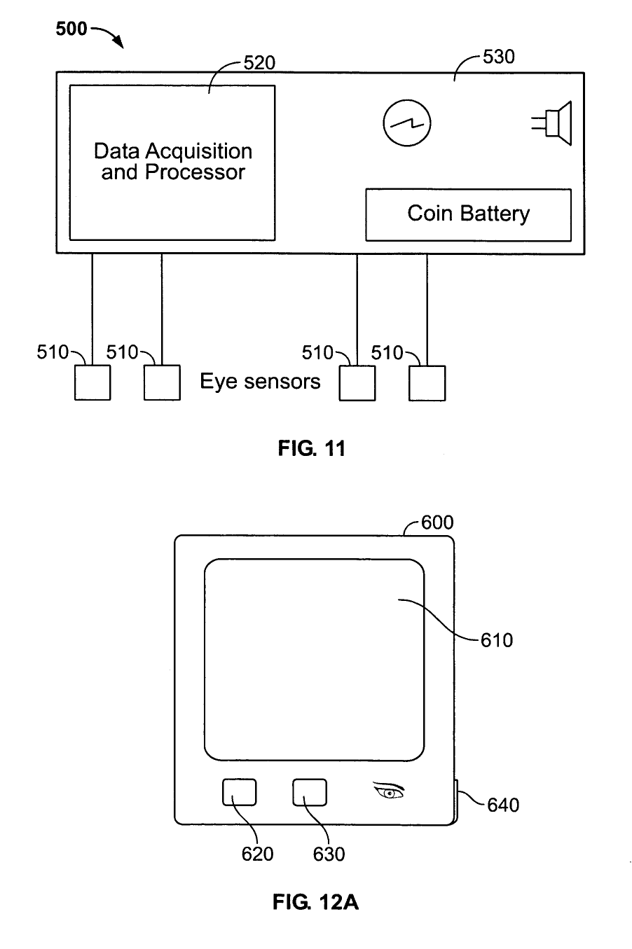

In this aspect, a medical operative or peri-operative monitoring system is also disclosed. Examples of such a system are provided in more detail below with respect to FIGS. 11-15. The system is configured for continuous real-time non-invasive quantitative monitoring of sources of direct external trauma (as described above) during prone or lateral patient positioning under general anesthesia for non-ophthalmic surgical procedures. The mounted transducers may be disposable. The system may include a removed or attached monitor display of measured parameters. The display may include an integrated alarm system by which a monitoring health care professional may be audibly and/or visually and/or tactilely (i.e. vibration) alerted in the setting of increased patient risk. The display may be disposable or reusable. The wire leads connect the eyelid-mounted transducers to the monitor. The monitor can be positioned to be monitored by the appropriate anesthesia team, monitoring team, or other designated health professional. Sources of external direct trauma may thus be monitored to further minimize the related potential for patient peri-orbital tissue complication(s) during a surgical procedure, particularly non-ophthalmic procedures.

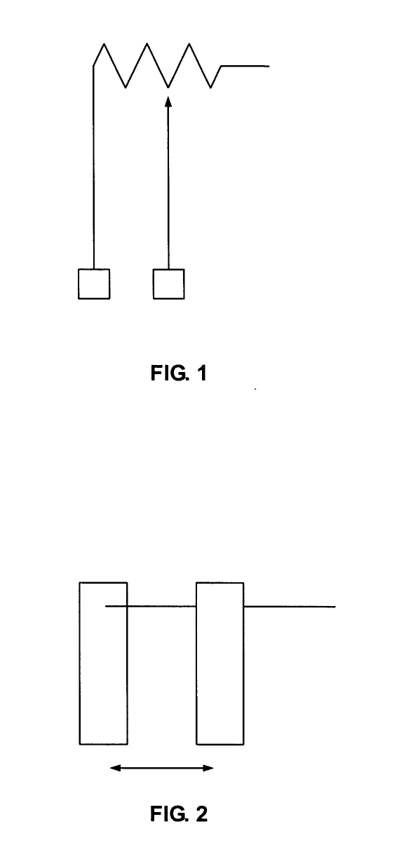

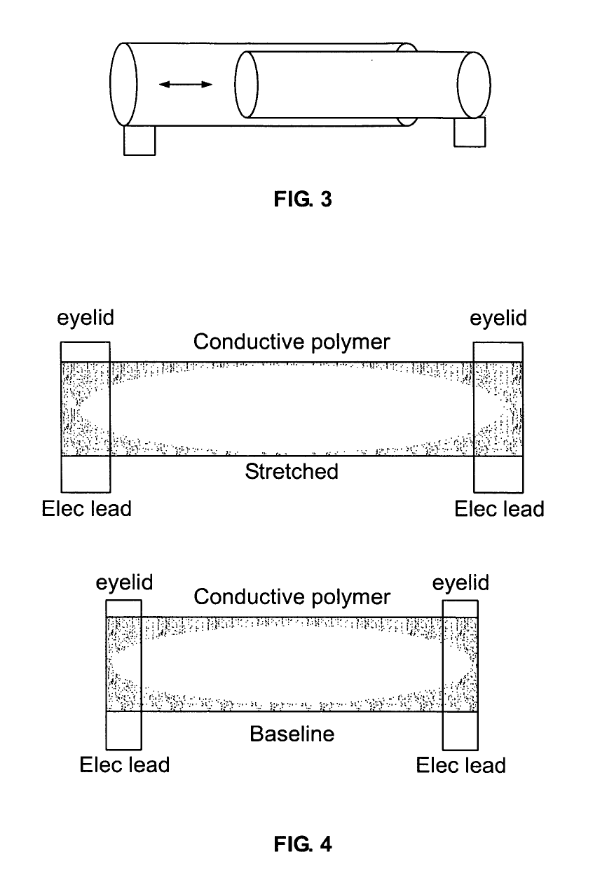

In another aspect, a method of monitoring for and against peri-orbital trauma of an individual during a surgical procedure includes the step of providing a peri-orbital external trauma monitoring system. This monitoring system can include one or more pressure sensors, each including one or more transducers, wherein each transducer is connected telemetrically or by a wire lead to a monitor. The method further includes the step of applying each of the transducers (alone or integrated into a mounting medium or device) to the superior eyelid and/or inferior eyelid and/or peripheral external peri-orbital tissues of the individual prior to the surgical procedure. The method further includes the step of placing the individual in a prone position. The method also includes performing a surgical procedure while monitoring the peri-orbital trauma monitoring system. The generated signals can be converted to an electrical signal that, when captured and analyzed, provides feedback to the healthcare provider through visible or audible means as an indicator of ocular and peri-orbital pressure and potential related visual loss. Examples of sensors are provided below with respect to FIGS. 1-10 and 13-15.

In this aspect, an ocular pressure sensor system measures externally applied normal and/or tangential forces acting on the patient's peripheral upper eyelid, lower eyelid, or adjacent external peri-orbital soft tissue (such as that proximal to the orbital rim) as sources of direct tissue trauma. Placement of these transducers towards the periphery of the peri-orbital region (as a measure of the external forces applied to these areas) provides specific information as to indirect pressure transfer to tissues within the osseous orbit/eye-socket (to include the optic musculature, the lacrimal nerve, the nasociliary nerve, and the accompanying vasculature and neurologic supply to these intra-orbital extra-ocular structures). The transducer system can be based on mechanical, chemical, or optical changes in properties, and can include the use of commercially available MEMS-fabricated piezo-resistive pressure sensor dies (see FIGS. 1-4 for examples of such transducer systems).

This method will provide non-averaged point-specific measure of applied force. The system can utilize a single point of force transduction or multiple points of transduction. For example, the system can include an ordered array of force transducers being arranged in a predetermined pattern. One pattern can include two transducers in which one of them is placed on a superior eyelid while another is placed on an inferior eyelid. Another ordered array can include three or more transducers covering the superior eyelid, inferior eyelid, and regions that extend laterally of the eye to cover the osseous orbital rim and other peripheral anterior peri-orbital tissue.

The generated signal can be converted to an electrical signal that, when captured and analyzed, can provide feedback to the healthcare provider through visible or audible means. When transduced by an array, the non-averaged forces may be processed and dimensionally mapped to provide interpretive static or dynamic information as to the force, magnitude, direction, location and character of the traumatic insult. Information in this regard may be expressed alphanumerically, graphically, audibly, tactilely, or in combination (to assist in determination of the relative risk of delivered force in given sets of force parameters (i.e. shear force versus point impact versus blunt trauma versus combination mechanisms). This system is characterized further by its use of point-specific transducers of sufficiently small size, accuracy, and response linearity. Any transducer system with specifications concordant with the above may be employed. Tissue temperature may be measured as well as an adjunctive indicator of adjacent tissue vascular or gross temperature compromise (which may predispose living tissue as more susceptible to the devitalizing effects of direct mechanical trauma).

Another system described involves monitoring peri-orbital edema during prone non-ophthalmic surgery as a measure of ocular pressure, and as a more broad measure of the risk of visual loss/blindness in this setting. The clinical setting in which blindness may occur is complex, multi-factorial, and includes factors such as increased intra-ocular and extra-ocular pressure. Given present understanding of the multiple causes of blindness after prone surgical positioning, the breadth of causes would be incompletely assessed by measures limited to direct external force to the eye or the central superior eyelid covering the eye. A more broad system of monitoring extra-ocular pressure, intra-ocular pressure and peri-orbital pressure is described here (with specifications most specific to use in the setting of prone position non-ophthalmic surgery, though it may be utilized in other settings if appropriate). This system detects/measures peri-orbital soft tissue edema as a measure of increased intrinsic peri-orbital tissue tension as well as both intra-ocular and extra-ocular pressure changes. These changes may produce external pressure on the eye, internal pressure changes within the eye, decreased vascular flow and decreased perfusion pressure to the optic nerve, retina and the entirety of the structures/tissues contained within the osseous orbit. Peri-orbital edema may also cause anterior eye displacement, described as a contributing factor in compartment syndrome of the optic nerve sheath (as outlined previously). Increasing peri-orbital edema may additionally predispose a patient to developing central retinal artery thrombosus or central retinal artery occlusion secondary to the perfusion pressure effects and other effects.

Therefore, peri-orbital edema may be monitored to simultaneously measure/assess a broad range of the diverse factors which may all lead to pressure-related eye injury, extra-ocular intra-orbital tissue compromise and/or visual loss/blindness as related to all of the above. Ocular and peri-orbital edema may be monitored/measured by multiple means to include optical, mechanical, and chemical methods.

In this aspect, a medical operative or peri-operative monitoring system is disclosed. The system is configured for continuous real-time non-invasive quantitative monitoring of peri-orbital edema/ocular pressure during prone or lateral patient positioning under general anesthesia for non-ophthalmic surgical procedures. The system can include one or more transducers. In one embodiment, the system can include a pair of transducers which can be positioned directly to each of a patient's eyelids prior to final patient prone or lateral surgical positioning for non-ophthalmic surgery. The transducers may be telemetric or can include attached leads, all of which may be mounted in a low profile, adhesive, material mounting. The mounted transducers and/or integrated wire leads can be disposable. The system can include a removed or attached monitor display of measured parameters. The display can include an integrated alarm system by which a monitoring health care professional may be audibly and/or visually and/or tactilely alerted in the setting of increased patient peri-orbital edema/ocular pressure. The display can be disposable or reusable. The wire leads can connect the eyelid-mounted transducers to the monitor. The monitor can be positioned to be monitored by the appropriate anesthesia team, monitoring team, or other designated health professional. Changes in peri-orbital edema may thus be monitored to further minimize the related potential for patient ocular complication(s) during a surgical procedure, particularly non-ophthalmic procedures. Peri-orbital edema may be measured as a directly related function of peri-orbital and/or eyelid cutaneous surface length change or elongation.

In one aspect, a method of monitoring ocular and/or peri-orbital edema of an individual during a surgical procedure includes the step of providing a peri-orbital edema monitoring system. The peri-orbital edema monitoring system can include two or more transducers, wherein each transducer is connected with a wire lead to a display and microprocessor unit. The method further includes the step of applying each of the transducers (alone or integrated into a mounting medium or device) to the eyelids and/or immediately adjacent external peri-orbital tissues of the individual prior to the surgical procedure. The method further includes the step of placing the individual in a prone position. The method also includes performing a surgical procedure while monitoring the display and microprocessor unit. The transducer system can be based on changes in mechanical, chemical, or optical properties. The generated signals can be converted to an electrical signal that, when captured and analyzed by the microprocessor, provides feedback to the healthcare provider through visible or audible means in the display unit as an indicator of ocular and peri-orbital pressure and potential related visual loss.

In a modified version of this method, both eyes of the patient are monitored simultaneously to measure and detect anomalies or significant differences in peri-orbital pressure or edema in the two eyes. Any of the sensors described herein may be used. Data from both eyes is processed at the same time, and both sets of data are analyzed by the microprocessor. The two sets of data are compared to one another by the microprocessor to assess the risk of peri-orbital visual compromise.

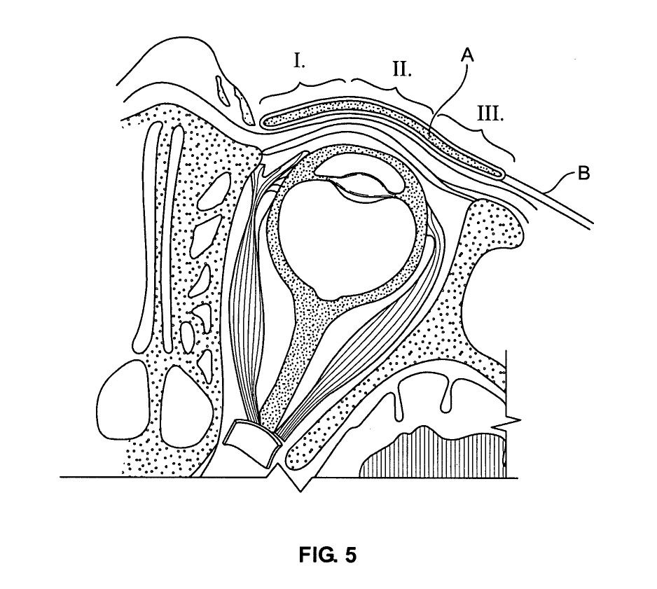

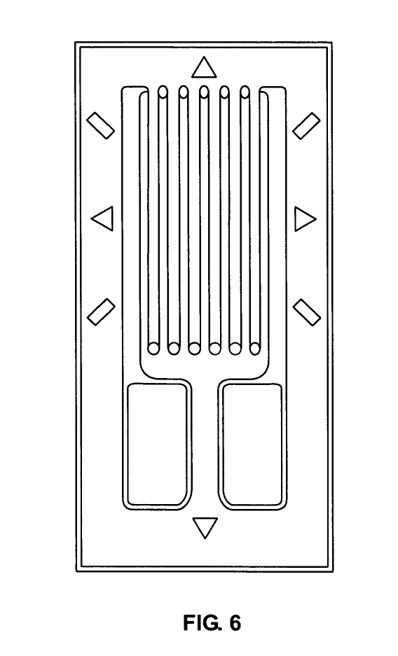

One embodiment provides a measure of superior eyelid and/or inferior eyelid changes in surface length as it develops in direct relation to edematous peri-orbital tissue change. As shown in FIGS. 4 and 5 and described in more detail below, this is performed by use of a transducer utilizing a conductive polymer chemical matrix that may be applied in direct proximity to the extra-ocular peri-orbital tissue surface of interest (to measure changes in surface length as described). In addition to this transducer, the transducer may be of any other known type to achieve the same end.

In a further embodiment, some or all of the transducing elements may operate telemetrically, which would eliminate concerns relative to the wires in proximity to adjacent soft tissue during travel to the monitor/processing unit.

In another embodiment, the transducer signals are transmitted by a single wire or multiple wires arranged as flat ribbon with a material covering of such property as to minimize soft tissue trauma in areas of potential patient contact.

In general the result of any alarm condition will be to alert the anesthesiologist, surgeon, monitoring team or other appropriate heath professional to assess the patient's eyes, peri-orbital tissues, and overall physiologic status in order to intervene in a manner to alter the risk factors precipitant to either mechanical trauma, increased per-orbital/ocular edema, or both.

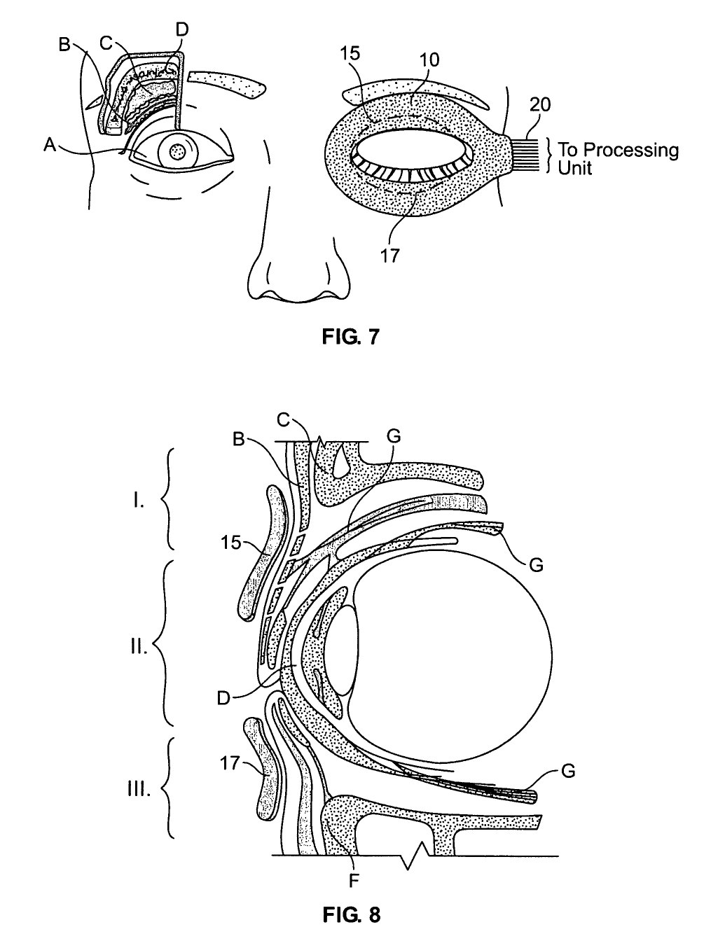

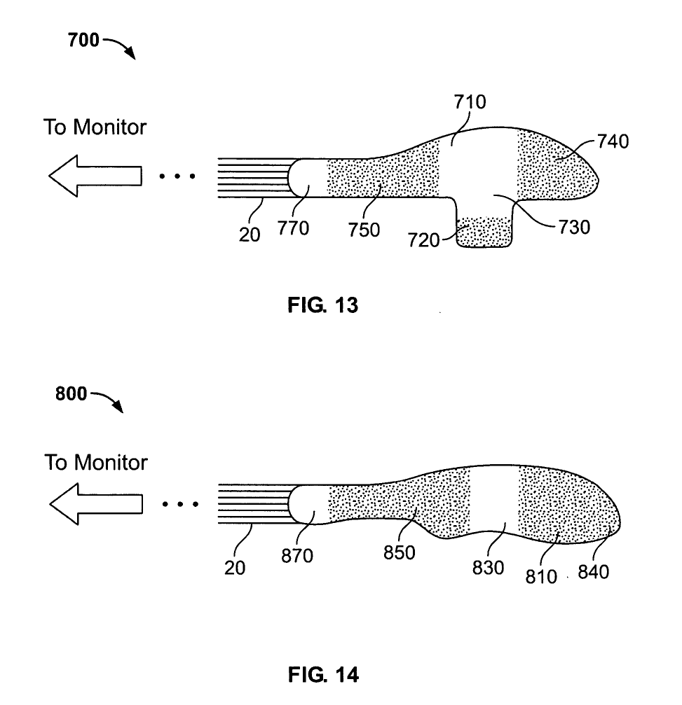

The systems described herein may include eyelid pads that integrate and position the transducer elements in a manner consistent with that described above. The eyelid pads may be made of various materials, selected and configured to minimize potential trauma/irritation to the tissues to which they are proximate. They are of low profile, though may otherwise be of varying shape/morphology, including those shown in FIGS. 7-10 and 13-15. They are also of specific design and material property so as to minimize anterior translation/subluxation of the eye during prone surgery (as precipitated by combined effects of gravity, anesthetic peri-orbital muscle relaxation, and anterior translational forces generated by posterior peri-orbital tissue edema). This is performed to minimize consequent tension on the optic nerve to therefore prevent optic nerve injury and visual loss by this mechanism (as may occur in the setting of compartment syndrome of the optic nerve sheath or other mechanisms). The eyelid pads may be further characterized by one of several unique adhesive patterns, such as those shown in FIGS. 13-15, that optimize transducer measures of desired parameters in desired locations while simultaneously effecting a mechanically supportive function to prevent anterior eye subluxation. Supportive materials that may be utilized in this function include (though are not limited to) formed structural foam(s).

Turning more specifically to the drawings, FIG. 1 depicts one embodiment of a peri-orbital edema/ocular pressure sensor. The sensor depicted in FIG. 1 shows a direct measurement of elongation through electrical resistance measurement. The sensor is a transducer that has one leg fixed to a resistive material while the other leg moves across the resistive material. This potentiometric arrangement allows the measurement of elongation through a change in electrical resistance. The resistive membrane can be screen printed on a flexible surface, such as polyester or polyimide, with etched electrical contacts. The mechanical assembly can be one of several arrangements such as those shown in FIGS. 2 (linear assembly) and 3 (radial assembly).

FIG. 4 shows another swelling/edema or pressure transducer embodiment. The sensor depicted in FIG. 4 includes a transducer that utilizes a chemically impregnated polymer that can measure electrical resistance. A conductive polymer is fabricated by mixing a conductive material (carbon black, metallic particles) in a polymer matrix (e.g., hydrogel). As the polymer gets stretched, the conductive properties of the polymer are altered allowing measurement of elongation through resistive changes. Mixing ratios of conductive material and aspect ratios of polymer matrix can define the relationship between electrical and mechanical forces. FIG. 5 is an illustration of an axial cross section of the entire orbit and eye depicting the peri-orbital edema/ocular pressure sensor depicted in FIG. 4 applied to the eye.

FIG. 6 is an illustration of a strain gage used to detect mechanical displacement in a peri-orbital edema/ocular pressure sensor. The strain gage can be used in any of the embodiments of a peri-orbital edema/ocular pressure sensor described herein.

FIG. 7 is an illustration of a peri-orbital sensor mounting 10 demonstrating desired peripheral placement of transducer(s) particular for monitoring of peri-orbital trauma. The mounting 10 includes a pressure sensor that is in electrical communication through leads 20 with a processing unit. The pressure sensor can be of any type including those described in FIGS. 1-4. The mounting 10 is oval in shape with a central cut-out portion to accommodate the eye. The central cut-out portion separates the superior transducer mounting 15 from the inferior transducer mounting 17 and exposes a substantial portion of the superior eyelid.

The illustration in FIG. 7 demonstrates peri-orbital coronal-section anatomy of the contralateral peri-orbital region. This provides clarification that the demonstrated embodiment is located peripherally to overlie the osseous orbital rim and soft tissues adjacent and peripheral to the eye itself. The anatomic dissection view of the contralateral peri-orbital region also provides demonstration of relevant anatomic structures of concern. Line ‘A’ identifies the globe or “eye” itself as located within the osseous orbit. Line ‘B’ identifies a cross-sectional view of the superior orbital musculature located adjacent to the eye/globe within the osseous orbit. Line ‘C’ identifies the superior lacrimal gland with surrounding peri-orbital adipose tissue, again located within the osseous orbit. Line ‘D’ identifies a cut-away section of the superior osseous orbital rim.

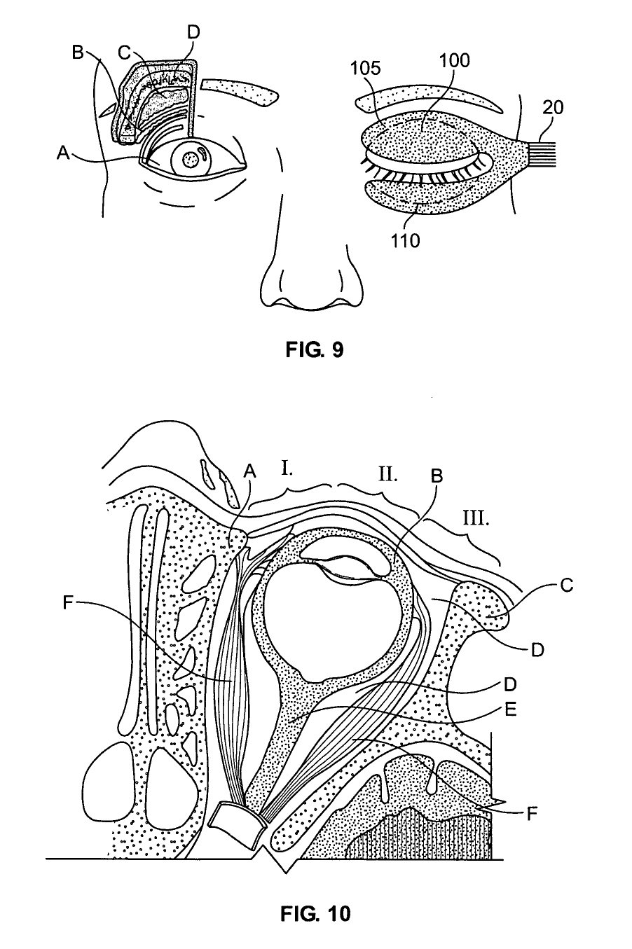

FIG. 8 an illustration depicting a sagittal cross section of the orbit and eye with inclusion of cross section of mounting 10. As shown, the eye is surrounded by various soft tissues, to include the optic musculature, intra-orbital nerves, arteries, veins, the optic nerve, connective and adipose tissue. The entirety of these tissues (to include the eye) are contained within the margins of the osseous orbit (laterally, inferiorly, superiorly, and posteriorly) and the superior and inferior conjunctiva and eyelid complexes (anteriorly). Additionally depicted is a cross section of sensor mounting 10 as most specific to the monitoring of peri-orbital trauma (as described previously). Three anatomic zones are identified in this figure (as represented by Roman numerals I, II, and III. Zone I denotes the superior aspect of the orbit, to include all superior extra-ocular intra-orbital anatomic contents, with inclusion of the superior half of the orbital rim. Zone II denotes the anatomic area overlying the globe, or eye itself. Zone III denotes the inferior aspect of the orbit, to include all inferior extra-ocular intra-orbital anatomic contents, extending peripherally to include the inferior half of the orbital rim. The mounting 10 shown in the figure is relegated preferentially to Zones I and II, peripheral to the eye. Mountings for the monitoring of peri-orbital edema (not shown) would overly any or all of zones I, II and III (as concordant with that shown in FIG. 9). As shown in FIG. 8, superior transducer mounting 15 of the peri-orbital trauma sensor is separated from the inferior transducer mounting 17 by a central cut-out portion, which exposes most of zone II. Line ‘B’ identifies the superior tarsal plate of the eyelid and the associated superior tarsal musculature. Line ‘C’ identifies the superior margin of the (osseous) orbit. Line ‘D’ identifies the anterior aspect of the eye, located centrally within the confines of the osseous orbit. Line ‘F’ identifies the osseous margin of the inferior orbit, the inferior orbital rim.

FIG. 9 is an illustration of a peri-orbital edema/ocular pressure sensor mounting 100 demonstrating an embodiment as particular for the monitoring of peri-orbital edema/ocular pressure. The mounting 100 includes a pressure sensor that is in electrical communication through leads 20 with a processing unit. The pressure sensor can be of any type including those described in FIGS. 1-4. The mounting 100 has two arms; a superior arm 105 and an inferior arm 110. The superior arm 105 covers most of the superior eyelid, while the inferior arm 110 covers a region just below the eye or the inferior eyelid. The two arms are separated by a cut-out portion. This shape allows a near-entirety or substantially all of the peri-orbital region to be addressed, with extension peripherally to include the osseous orbital rim (circumferentially in a coronal plane). Line ‘A’ identifies the globe or “eye” itself as located within the osseous orbit. Line ‘B’ identifies a cross-sectional view of the superior orbital musculature located adjacent to the eye/globe within the osseous orbit. Line ‘C’ identifies the superior lacrimal gland with surrounding peri-orbital adipose tissue, again located within the osseous orbit. Line ‘D’ identifies a cut-away section of the superior osseous orbital rim. The illustration in FIG. 9 demonstrates peri-orbital coronal cross section anatomy of the contralateral peri-orbital region. The mounting 100 is positioned centrally over the area of the eye itself and covers the superior and inferior eyelids. The area coverage/transduction may extend to overly any or all of the peri-orbital region.

FIG. 10 is an illustration depicting an axial cross section of the entire orbit and eye. Three anatomic zones are identified in the anatomic plane of this figure (zones I, II, and III). Zone I includes the intra-orbital extra-ocular structures medial to the eye, extending peripherally to include the orbital rim. Zone II includes the anterior area overlying the eye/globe itself. Zone III includes the intra-orbital extra-ocular structures lateral to the eye, extending peripherally to include the lateral osseous orbit (orbital rim). All three areas are preferred for the purpose of peri-orbital edema.ocular pressure monitoring, while peripheral areas I and III are preferred areas for the purpose of peri-orbital trauma monitoring. Line ‘A’ identifies the medial aspect of the osseous orbital rim. Line ‘B’ identifies the anterior margin of the eye. Line ‘C’ identifies the lateral aspect of the osseous orbital rim. Line ‘D’ identifies an area of extra-ocular intra-orbital adipose tissue, adjacent to the peripherally located intra-orbital nerves, vessels, and musculature. Line ‘E’ identifies the segment of the optic nerve located within the confines of the osseous orbit. It is located peripheral and posterior to the eye as shown (in direct proximity to the remainder of the extra-ocular contents of the osseous orbit). Line ‘F’ identifies the medial and lateral orbital musculature.

FIG. 11 is a diagram of an integrated peri-orbital trauma and peri-orbital edema/ocular pressure sensor system 500 for use in monitoring related safety during surgical procedures, particularly non-ophthalmic surgical procedures in the prone position. The system include a sensor assembly 510 for each eye. Each sensor assembly 510 can include one or more transducers. In one embodiment, each sensor assembly 510 includes an array of transducers that is arranged in a predetermined pattern that covers the superior eyelid and inferior eyelid. Such an assembly can have one transducer for the superior eyelid and one for the inferior eyelid, or more than one transducer for each of the superior and inferior eyelids. In another embodiment, each sensor assembly 510 includes an array of transducers that is arranged in a predetermined pattern that covers the superior eyelid, inferior eyelid and the osseous orbital rim of the eye. Any of the sensors described herein can be used to form the sensor assembly 510, such as sensors 10, 100, 700, and 800, depicted in FIGS. 7, 9, 13 and 14 respectively.

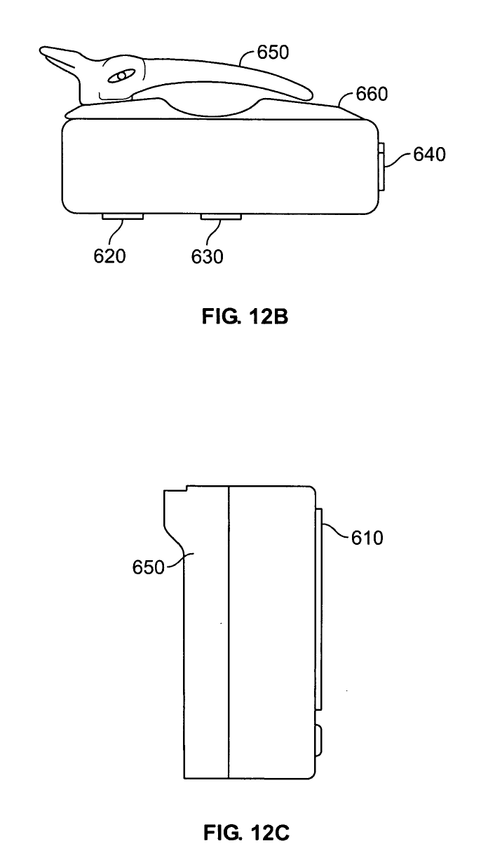

FIGS. 12A, 12B and 12C depict a monitor 600 associated with a peri-orbital edema/ocular pressure or peri-orbital trauma monitoring system. The monitor 600 includes a display 610, an on/off switch 620, and an alarm silencer 630. The monitor also includes ports 640 for connection with leads from a peri-orbital edema/ocular pressure sensor or a peri-orbital trauma sensor such as those described herein. The monitor 600 includes a hinge clip 650 for attachment to an IV pole. The monitor 600 also has a rubberized backing surface 660 to maintain traction on an IV pole. The monitor 600 can be disposable or reusable. The display 610 can depict pressure and edema data for each eye separated into two separately colored columns. The sensor mounts (e.g., sensor mounts 10 or 100) for each eye can be a separate color each with a correspondingly colored lead. The input ports 640 can have corresponding colors that match the colors of the sensor mounts. This coloring system can make it easy for the surgical team to quickly and easily identify the eye associated with the data depicted in the monitor 600.

FIGS. 13 and 14 show other shapes and geometries of peri-orbital edema/ocular pressure sensors. FIG. 14 depicts a sensor 800 having a superior lid region 810 with one or more leads 20 for connection to a monitor, such as the monitor 600 described above. FIG. 13 depicts a sensor 700 having a superior lid region 710 a tail 720 to keep the eyelid closed during surgery. In both sensors 700 and 800, the superior lid regions 710 and 810 have an adhesive pattern such that lateral portions 740/840 and 750/850 respectively of the lid regions include adhesive material while central gap portions 730/830 have no adhesive. The gap portions 730/830 provide a span across which the eyelid skin strain/swelling can be transduced. The superior lid regions 710 and 810 of sensors 100 and 800 can be made of an elastic foam that stretches to accommodate for swelling and edema. Each sensor 700 and 800 also includes a respective tail portion 770 and 870 that protects the delicate skin immediately adjacent to the center of the eye. Either the tail portions 770/870 or the entirety of the sensors 700/800 can include various anti-biotic and/or anti-septic agents.

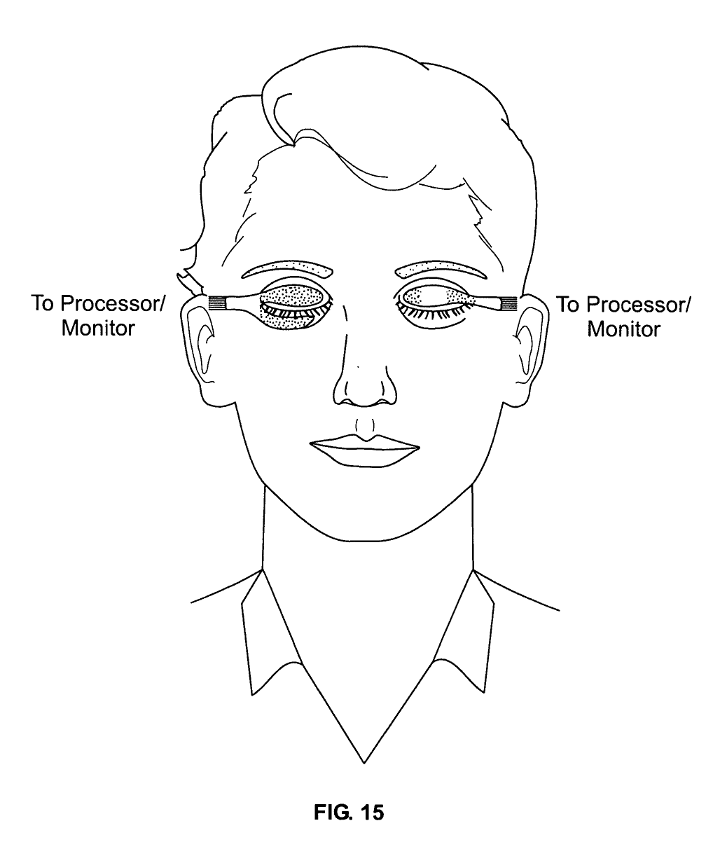

FIG. 15 is an illustration of two eyelid pads/sensor mountings and leads for transduction of signals representing peri-orbital edema/ocular pressure, each with a different geometry applied to a patient. Thus, a number of different geometries can be used to both monitor various peri-orbital regions while simultaneously mechanically supporting and protecting the globe as part of an integrated approach.

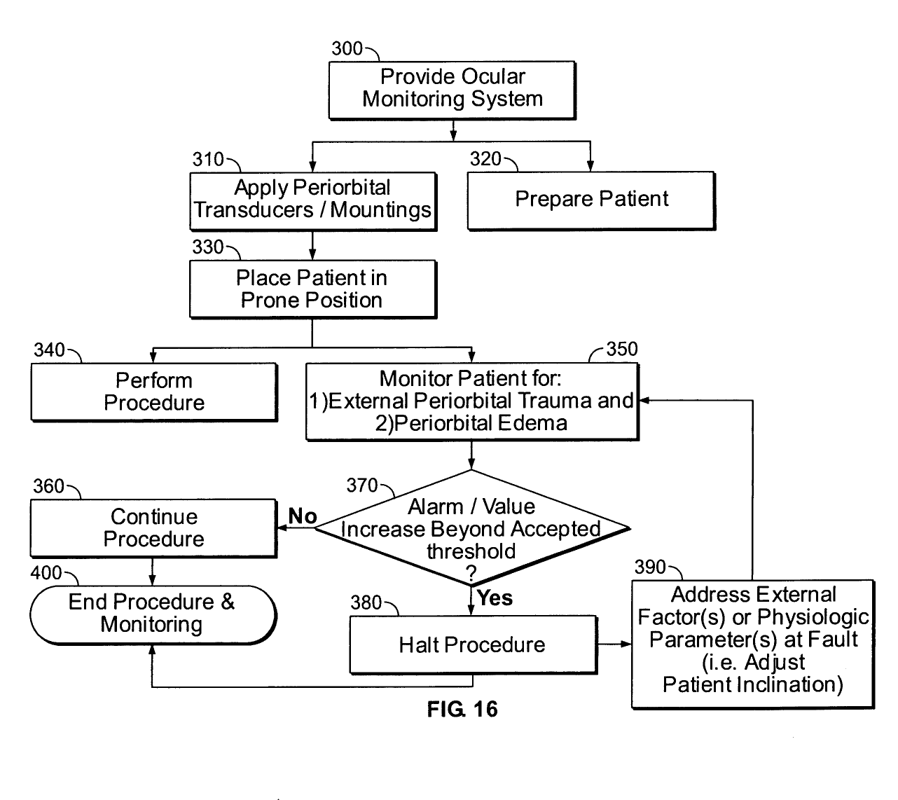

FIG. 16 is a flowchart depicting a method of monitoring a patient for peri-orbital trauma and/or periobital edema/ocular pressure. The method includes providing a peri-orbital edema/ocular pressure monitoring system at 300. The peri-orbital edema/ocular pressure monitoring system includes a pair of transducers, one for each eye. The transducers are applied to the patient at 310 while the patient is being prepared for surgery at 320. The patient is then placed in a prone position for the surgical procedure at 330. Using the peri-orbital edema/ocular pressure monitoring system the patient is monitored at 340 while the surgical procedure is performed at 350. The peri-orbital edema/ocular pressure monitoring system monitors the patient's peri-orbital edema and ocular pressure during surgery and if the measured parameters exceed a predetermined threshold at 360, the surgical procedure is halted at 365 and the causative factor is addressed to reduce risk of visual loss at 370. The patient is continuously monitored at 360 and when the peri-orbital edema/ocular pressure reading is reduced below the predetermined threshold, the surgical procedure can be continued at 380 until it is completed and terminated at 390. If there is no reduction in peri-orbital edema/ocular pressure, the surgical procedure may be abandoned and terminated at 390. An identical algorithm may be applied as it pertains to monitoring and measure of direct external peri-orbital mechanical forces.

While the invention and methods herein disclosed have been described by means of specific embodiments and applications thereof, numerous modifications and variations could be made thereto by those by those skilled in the art without departing from the scope of the invention set forth in claims.

1. A method of monitoring a patient's eyes during a surgical procedure comprising:
applying a first pressure sensor to a first eye of the patient, wherein the pressure sensor is applied to a peripheral superior eyelid, an inferior eyelid, or other peri-orbital tissue such that forces directed at the patient's anterior peri-orbital tissue are detected; and
transmitting a first set of data representative of the detected forces to a display; and
monitoring the display during the surgical procedure.

2. The method of claim 1, further including the step of placing the patient in a prone position prior to performing the surgical procedure.

3. The method of claim 1, further comprising:
applying a second pressure sensor to a second eye of the patient, wherein the second pressure sensor is applied to a superior eyelid, an inferior eyelid, or other peri-orbital tissue such that forces directed at the patient's anterior peri-orbital tissue are detected; and
transmitting a second set of data representative of the detected forces to the display; and
monitoring the display during the surgical procedure.

4. The method of claim 3, further comprising comparing the first set of data to the second set of data during the surgical procedure to assess the risk of peri-orbital visual compromise.

5. A peri-orbital trauma monitoring system comprising:
a pressure sensor capable of measuring external applied loads to peripheral anterior peri-orbital tissue;
a microprocessor that is capable of processing information received from the sensor;
communication means between the sensor and microprocessor;
a display in communication with the microprocessor;
a sensor mount that is shaped to attach the sensor to peri-orbital tissue.

6. The peri-orbital trauma monitoring system of claim 5, wherein the pressure sensor comprises one or more transducers that measure non-averaged point specific gradients of applied external forces.

7. The peri-orbital trauma monitoring system of claim 5, wherein the pressure sensor comprises an array of transducers that are arranged in a predetermined pattern to measure an array of forces, wherein the predetermined pattern includes at least one transducer adjacent to each of the superior eyelid and inferior eyelid.

8. The peri-orbital trauma monitoring transducer system of claim 7, wherein the array of transducers measures one or more dimensional parameters and the microprocessor processes data received from the array and provides an output that characterizes the forces measured by the array and thereby provides a relative risk of peri-operative visual compromise as a result of the applied external forces.

9. The peri-orbital trauma monitoring system of claim 8, wherein the one or more dimensional parameters comprise magnitude, direction, and rate of change of force.

10. The peri-orbital trauma monitoring system of claim 5, wherein the means for communication between the sensor and microprocessor comprises a telemetric signal and electronics for generating and receiving said signal.

11. The peri-orbital trauma monitoring system of claim 5, wherein the means for communication between the sensor and microprocessor comprises one or more leads connecting the sensor and microprocessor.

12. The peri-orbital trauma monitoring system of claim 5, wherein the sensor is adapted to be mounted away from the eye at the periphery of an eye orbit to measure external force as a measure of non-ocular peri-orbital tissue pressure.

13. The peri-orbital trauma monitoring system of claim 12, wherein the microprocessor provides an output representative of peri-orbital tissue pressure as a measure of transmitted ocular pressure and optic nerve pressure.

14. The peri-orbital trauma monitoring system of claim 5, wherein the sensor measures external force as a measure of non-ocular peri-orbital tissue pressure and the microprocessor provides an output representative of peri-orbital tissue pressure as a measure of transmitted ocular pressure and optic nerve pressure.

15. A method of assessing ocular pressure comprising:
providing an ocular pressure monitoring system;
using the ocular pressure monitoring system to monitor peri-orbital edema during prone positioning of a patient; and
performing a surgical procedure while monitoring peri-orbital edema.

16. The method of claim 15, wherein the ocular pressure monitoring system comprises:
a first transducer mounted in a material mounting that attaches to peri-orbital tissue of a first eye of a patient;
a second transducer mounted in a material mounting that attaches to peri-orbital tissue of a second eye of a patient; and
a microprocessor that is in communication with the first and second transducers, wherein said microprocessor processes data received from the transducers; and
a display that displays the data.

17. The method of claim 16, wherein the peri-orbital tissue of the first eye and second eye comprises the superior eyelid and inferior eyelid respectively of each of said first and second eyes.

18. The method of claim 15, further including the step of placing the patient in a prone position prior to performing the surgical procedure.

19. An ocular pressure monitoring system comprising:
a plurality of transducer assemblies that measure peri-orbital edema from peri-orbital tissue areas associated with one or both of a patient's eyes, each of said transducer assemblies comprising at least one pressure transducer and at least one mounting that is shaped to secure the transducer to at least one peripheral peri-orbital tissue area;
a plurality of leads wherein each of the leads is attached to one of the transducers;
a microprocessor connected to the transducers by the leads; and
a display unit in communication with the microprocessor for displaying data representative of peri-orbital edema.

20. The ocular pressure monitoring system of claim 19, wherein the transducers measure change in tissue surface length using optical, mechanical, electrical, or chemical means.

21. The ocular pressure monitoring system of claim 19, wherein the transducers measure change in tissue surface length using measured change in the electrical resistivity of a conductive polymer matrix.

22. The ocular pressure monitoring system of claim 19, wherein the display unit provides visible, audible, or tactile indicators to monitoring personnel.

23. The ocular pressure monitoring system of claim 19, wherein the mountings comprise a shape, adhesive pattern, and material property that supports the eye against anterior translation during prone positioning.

24. The ocular pressure monitoring system of claim 19, wherein the transducers measure non-averaged point specific gradients of applied external forces on the peri-orbital tissue areas.

25. The ocular pressure monitoring system of claim 19, wherein the force sensor comprises an array of transducers that are arranged in a predetermined pattern to measure an array of forces, wherein the predetermined pattern includes at least one transducer adjacent to each of a superior eyelid and an inferior eyelid of each eye.

26. The ocular pressure monitoring system of claim 25, wherein each of the array of transducers measures one or more dimensional parameters and the microprocessor processes data received from the array and provides an output that characterizes the forces measured by the array, thereby providing a relative risk of peri-operative visual compromise as a result of the applied external forces.

27. The ocular pressure monitoring system of claim 26, wherein the one or more dimensional parameters comprise magnitude, direction, and rate of change of force.

28. The ocular pressure monitoring system of claim 19, wherein at least one of the mountings has an oval shape comprising a superior mounting region adapted for attachment to a portion of a superior eyelid and an inferior mounting region adapted for attachment to an inferior eyelid, wherein a central cut-out portion separates the superior mounting region from the inferior mounting region and exposes a substantial portion of the superior eyelid.

29. The ocular pressure monitoring system of claim 19, wherein at least one of the mountings comprises:
a first arm with a proximal end and a distal end, the first arm adapted for attachment to a superior eyelid and sized to cover substantially all of the superior eyelid; and
a second arm with a proximal end and a distal end, the second arm adapted for attachment to an inferior eyelid,
wherein the two arms are connected at their proximal ends but not their distal ends, and wherein the two arms have a length that extends laterally of the eye to cover an osseous orbital rim of the eye.

30. The ocular pressure monitoring system of claim 19, wherein there are two transducer assemblies each with its own mounting, and wherein each of the two mountings has a shape that is different from the other such that the two transducers measure a different peri-orbital region.
